(19) United States
(12) Patent Application Publication
(10) Pub. No.: US 2026/0026972 A1
(43) Pub. Date: Jan. 29, 2026

(54) DRESSING WITH INTEGRATED SUPPORT FOR USE WITH A PERCUTANEOUS DEVICE

(71) Applicant: Cath Dry, Inc., Los Angeles, CA (US)

(72) Inventor: Paul Ryan, County Cavan (IE)

(21) Appl. No.: 18/784,115

(22) Filed: Jul. 25, 2024

(51) Int. Cl. A61F 13/00

(52) U.S. Cl. CPC A61F 13/00051

(57) ABSTRACT

A dressing for use at an exit site of a percutaneous device extending from the skin of a patient is disclosed. The dressing includes a dressing body with a periphery, a top surface, and an opposite bottom surface that is configured to contact the skin of the patient. The dressing further includes an opening that is configured to receive a portion of the percutaneous device through the dressing body and a slot that extends from the opening to the periphery of the dressing body. The dressing body includes an absorbent layer impregnated with an antiseptic compound. The dressing further includes a clasp disposed in the opening. The clasp is configured to engage the portion of the percutaneous device that extends through the opening to hold the dressing in place over the exit site.

### TECHNICAL FIELD

This application relates generally to medical dressings and specifically to a medical dressing for use at the exit site of a percutaneous device.

### BACKGROUND

The use of percutaneous devices, including long-term or vascular access catheters, such as a central venous catheter, is common in medical procedures. In many cases, these devices provide extended access to blood vessels, reducing the need for multiple punctures to obtain blood samples or administer medications. For example, a peripherally inserted central catheter (PICC) is a form of IV access that may be used for a prolonged period of time (e.g., for long chemotherapy regimens). The area where a catheter or similar percutaneous device enters or exits the skin is known as the exit site, which is particularly susceptible to infections or irritation due to it generally being an open wound. The area beneath the skin of the patient along the shaft of the catheter or other percutaneous device is referred to as the catheter tract.

Typically, the exit site for a long-term catheter, such as a PICC line, is covered with a dressing to protect against infection by forming a physical barrier that prevents bacteria, debris, or contaminants from entering the patient's body. Conventional dressings include an adhesive pad or surface to secure the dressing around the exit site and the device. The adhesive pad or tape may absorb moisture, dead skin cells, and bacteria, all of which promote infection instead of preventing it. Further, the adhesive pad or tape fall off over time, resulting in little to no protection for the exit site. The adhesives may also irritate the skin and require special cleaning agents to ensure proper adhesion of the dressing to the skin.

Therefore, there is a need for an adhesive-free dressing that can remain securely in place over the exit site of the percutaneous device, providing a barrier against bacteria and contaminants without causing undue irritation to the patient's skin.

### SUMMARY

In accordance with embodiments of the invention, a dressing for use at an exit site of a percutaneous device extending from the skin of a patient is disclosed. The dressing includes a dressing body with a periphery, a top surface, and an opposite bottom surface that is configured to contact the skin of the patient. The dressing further includes an opening that is configured to receive a portion of the percutaneous device through the dressing body and a slot that extends from the opening to the periphery of the dressing body. The dressing body includes an absorbent layer impregnated with an antiseptic compound. The dressing further includes a clasp disposed in the opening. The clasp is configured to engage the portion of the percutaneous device that extends through the opening to hold the dressing in place over the exit site.

According to one aspect of the invention, the bottom surface of the dressing body may be non-adhesive. Rather, the clasp is configured to hold the dressing in place over the exit site. In one embodiment, the clasp may extend about an inner wall of the dressing body defined by the opening. For example, the clasp may be configured to directly contact the portion of the percutaneous device that extends through the opening. Furthermore, the clasp may be configured to apply a force so as to grip the portion of the percutaneous device that extends through the opening.

According to another embodiment, the clasp may include a pair of legs. Each leg may be configured to extend partially along the slot in the dressing body. For example, the pair of legs may be spaced apart from each other to define an open space therebetween. The space between the pair of legs may be constant along a length of the slot, for example. In one aspect, each leg may include a web member that extends between a top flange and a bottom flange. Each web member may be configured to engage a radial edge that defines the slot in the dressing body. In another aspect, the percutaneous device may include a catheter line and the pair of legs may be configured to straddle the catheter line.

According to one aspect, the dressing body may comprise a film layer disposed on top of the absorbent layer. The film layer may form the top surface of the dressing body. In another aspect, the film layer may extend into the opening such that the film layer is arranged between the clasp and the portion of the percutaneous device that extends through the opening. In yet another aspect, the clasp may be coupled to the dressing body such that the top surface and the bottom surface of the dressing body are generally planar. In one aspect, the clasp may be coupled to the absorbent layer.

In one aspect, the clasp may include a web member that extends between a top flange and a bottom flange to form a channel. The dressing body may be received within the channel to couple clasp to the dressing body. For example, the top flange of the clasp may be configured to engage the top surface of the dressing body and the bottom flange may be configured to engage the bottom surface of the dressing body. In another aspect, the antiseptic compound may include Chlorhexidine. Additionally or alternatively, a thickness measured between the top surface and the bottom surface of the dressing body may be within a range of between about 0.1 cm to about 0.3 cm.

According to another embodiment, a method of applying the dressing according to any one of the aspects described above to the exit site of a percutaneous device that extends from skin of a patient is disclosed. The method includes positioning the percutaneous device in the slot and moving the dressing relative to the percutaneous device to receive the percutaneous device into the slot in a direction toward the opening in the dressing and positioning the bottom surface of the dressing body into engagement with the skin of the patient surrounding the exit site. Followed by receiving the percutaneous device within the clasp and engaging the percutaneous device with the clasp to hold the dressing in place over the exit site.

According to one aspect, the clasp may include a pair of legs and the slot in the dressing body may be defined by a pair of radial edges. Each leg may be configured to extend partially along one radial edge of the dressing body. The method may further include engaging the percutaneous device with the pair of legs to hold the dressing in place over the exit site.

The steps and elements described herein as part of various embodiments and aspects can be reconfigured and combined in different combinations to achieve the desired technical effects as may be desired. To this end, the embodiments and aspects can be combined in any combination or sub-combination.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitute a part of this specification, illustrate one or more embodiments of the invention and, together with the general description given above and the detailed description given below, serve to explain the one or more embodiments of the invention.

FIG. 1 is a perspective view of a dressing in use at an exit site of a percutaneous device in accordance with one embodiment of the present invention.

FIG. 2 is a top view of the dressing of FIG. 1, illustrating details of a clasp of the dressing.

FIG. 2A is a partial disassembled view of the dressing of FIGS. 1 and 2.

FIG. 3 is a top view of a dressing according to another embodiment of the present invention.

FIG. 4A is a schematic cross-sectional view of the dressing taken along line 4A-4A of FIG. 1, illustrating additional details of the clasp.

FIG. 4B is a view similar to FIG. 4A, illustrating additional details of a clasp of a dressing according to an alternative embodiment of the present invention.

FIG. 4C is a view similar to FIG. 4A, illustrating additional details of a clasp of a dressing according to an alternative embodiment of the present invention.

FIG. 5A is a view similar to FIG. 4A, illustrating additional details of a clasp of a dressing according to an alternative embodiment of the present invention.

FIG. 5B is a view similar to FIG. 4A, illustrating additional details of a clasp of a dressing according to an alternative embodiment of the present invention.

### DETAILED DESCRIPTION

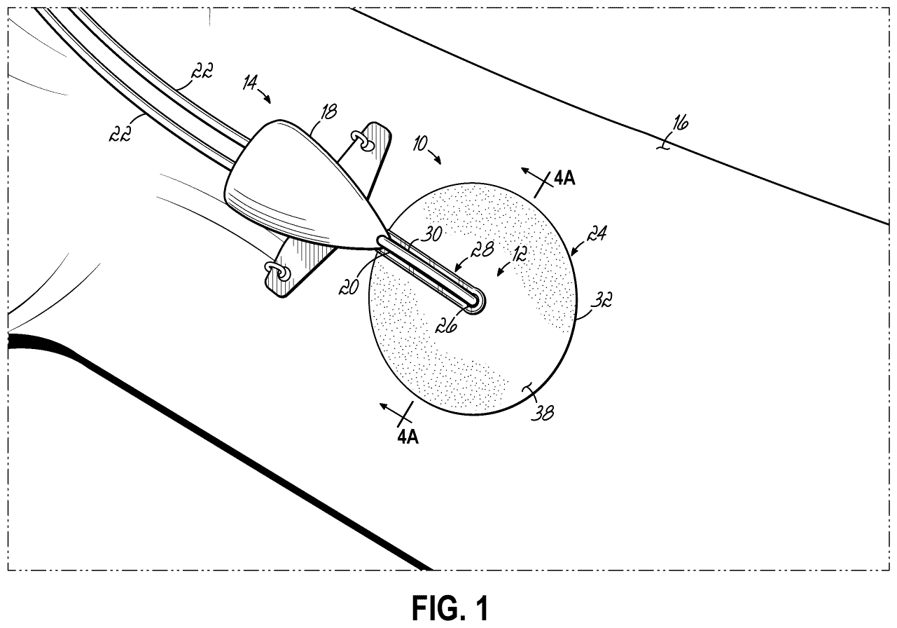

Referring now to the Figures, FIG. 1 illustrates a dressing 10 for use at an exit site 12 of a percutaneous device 14 extending from the skin 16 of a patient according to one embodiment of the present invention. The percutaneous device 14 may be a peripherally inserted central catheter (PICC), for example. In that regard, the percutaneous device 14 may include a hub 18 from which a catheter line 20 extends for insertion into the body of the patient at the exit site 12. In that regard, the exit site 12 is the area of the patient's skin 16 where the catheter line 20 enters or exits the body, for example. The percutaneous device 14 further includes a pair of dependent lines 22 that may define or include the additional lumens or channels that supply fluid to or from the catheter line 20. To that end, the percutaneous device 14 may include fewer or more dependent lines 22. While the dressing 10 is shown and described in use with a specific type of percutaneous device 14, it will be understood that the dressing 10 may be used with various percutaneous device designs without departing from the scope of the present invention.

The dressing 10 includes a dressing body 24 that is configured to contact the patient's skin 16, covering and extending over the exit site 12 and extending about the percutaneous device 14. In that regard, the dressing body 24 may be semi-rigid yet sufficiently flexible, allowing the dressing 10 to be comfortably worn without folding over itself, bunching, or otherwise causing discomfort to the patient's skin 16 and the exit site 12. As shown in FIG. 1, the dressing body 24 is generally circular in shape and includes a central opening or hole 26 through which a portion of the percutaneous device 14, such as the catheter line 20, is configured to extend for insertion into the body of the patient at the exit site 12. The opening 26 is configured to be positioned over the exit site 12, with the dressing body 24 extending generally circumferentially about the catheter line 20 of the percutaneous device 14. As will be described in further detail below, the dressing 10 includes a fastener in the form of a clasp 28 positioned within the opening 26. In that regard, the clasp 28 may define the opening 26 and is configured to engage a portion of the catheter line 20 of the percutaneous device 14 that passes through the opening 26, securely coupling the dressing 10 to the percutaneous device 14. The clasp 28 ensures that the dressing 10 remains positioned over the exit site 12, thereby providing a sterile barrier against bacteria and contaminants without the need for adhesives.

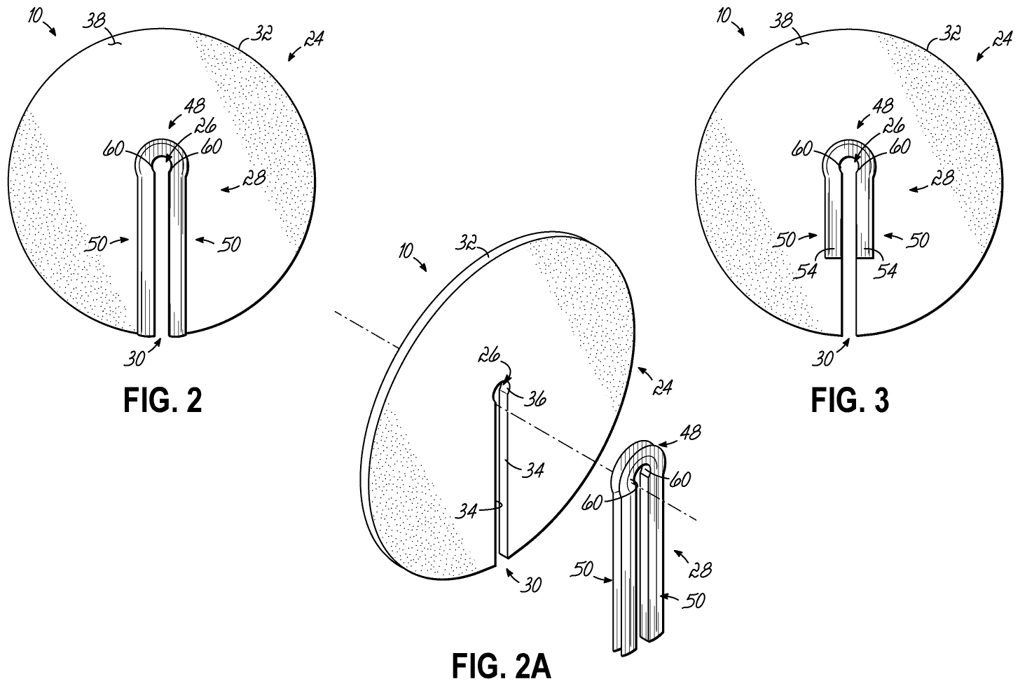

Referring now to FIGS. 1-2A, the dressing 10 includes a slot 30 formed in the dressing body 24. The slot 30 extends from the opening 26 in the dressing body 24 to a periphery 32 of the dressing body 24. In that regard, the slot 30 defines a channel or passageway through the dressing body 24 from the periphery 32 to the opening 26. As best shown in FIG. 2A, the slot 30 is defined by a pair of radially extending edges (“radial edges”) 34 that extend from an inner sidewall 36 that defines the opening 26 to the periphery 32 of the dressing body 24. The pair of radial edges 34 are spaced apart from each other to form an open space between them, being the slot 30. The space between the pair of radial edges 34 defines a width of the slot 30, being the distance between the radial edges 34. The width of the slot 30 may remain constant along a length of the slot 30 from the opening 26 to the periphery 32 of the dressing body 24, as shown. The width of the slot 30 may be less than a diameter of the catheter line 20, as will be described in further detail below.

The slot 30 formed in the dressing body 24 differs from a slit. For example, the slot 30 defines a measurable space between the radial edges 34 of the dressing body 24; a space devoid of material. In contrast, a slit is merely a narrow cut that does not create a measurable space between its edges; no material is removed to form a slit. Thus, the edges of a slit may remain in contact along a length of the slit. To that end, the slot 30 allows the dressing 10 to be easily applied about the percutaneous device 14 and removed without disrupting the position of the percutaneous device 14 or otherwise irritating the exit site 12. That is, the width of the slot 30 allows the catheter line 20 of the percutaneous device 14 to be received into the slot 30, and effortlessly moved along the slot 30 and into positioning with the opening 26. Little to no manipulation of the dressing 10, such as stretching or spreading of the dressing body 24, is needed to open the slot 30 to receive the catheter line 20 into the opening 26. This ensures an easy and hygienic application of the dressing 10 over the exit site 12. To that end, the clasp 28 may ensure that the radial edges 34 remain spaced apart, as will be described in further detail below.

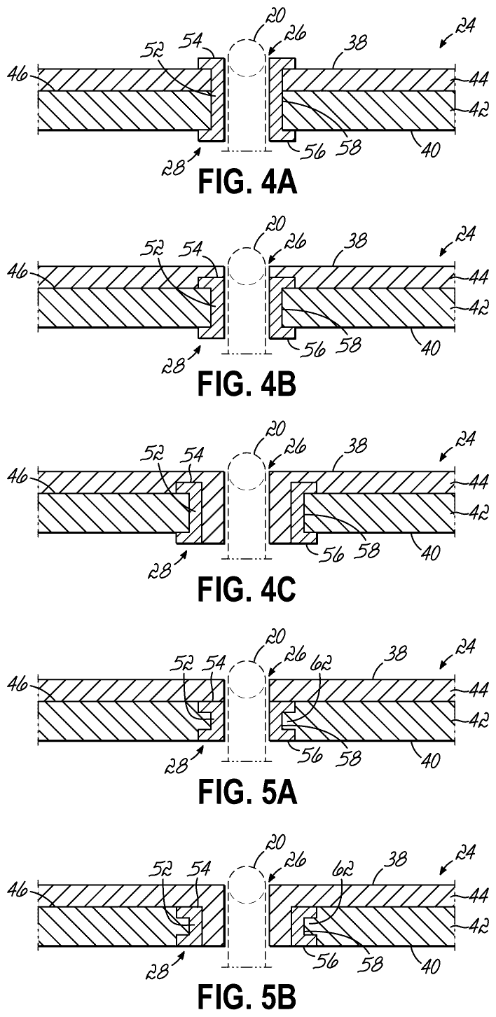

With reference to e.g., FIGS. 1 and 4A, the dressing 10 includes a top surface 38 and an opposite bottom surface 40 that is configured to contact the skin 16 of the patient. The top surface 38 and the bottom surface 40 may be generally planar. A thickness of the dressing 10, as measured between the top surface 38 and the bottom surface 40, may be within a range of between about 0.1 cm to about 0.3 cm, for example. In the embodiment shown, the dressing 10 includes an absorbent layer 42 that forms the bottom surface 40 of the dressing 10 and a film layer 44 disposed on a top surface 46 of the absorbent layer 42, the film layer 44 forming the top surface 38 of the dressing 10, as shown in e.g., FIG. 4A. The film layer 44 may be a coating disposed over the top 46 of the absorbent layer 42. To that end, the a thickness of the film layer 44 may be less than a thickness of the absorbent layer 42. Together, the film layer 44 and the absorbent layer 42 define the dressing body 24.

While the exemplary embodiment of the dressing 10 is shown as having including the film layer 44 and the absorbent layer 42, it will be understood that the dressing 10 may include fewer or more layers. In any event, the top surface 38 and the bottom surface 40 of the dressing 10 are non-adhesive. That is, the top surface 38 and the bottom surface 40 of the dressing 10 do not contain any adhesive to adhere the dressing 10 to another surface, such as the patient's skin 16. Instead, the clasp 28 is configured to positively engage the catheter line 20 of the percutaneous device 14 to maintain a position of the dressing 10 over the exit site 12, eliminating the need for adhesive on the bottom surface 40 of the dressing 10. As such, the dressing 10 is secured in place over the exit site 12 without using adhesive. Thus, the dressing 10 is held securely in place by the clasp 28 to form a hygienic physical barrier that prevents bacteria, debris, or contaminants from entering the patient's body. Yet, by not including adhesive on the bottom surface 40 of the dressing 10, the negative effects of adhesive on the skin 16, such as irritation to the skin 16 and the exit site 12, are avoided.

The absorbent layer 42 may include or be impregnated with an antiseptic and/or antimicrobial compound. For example, the absorbent layer 42 may be formed from a sponge or foam-like material, such as Synthetic Polymers, Polyurethane (PU) or Silicone, for example. Suitable antiseptic and/or antimicrobial agents include a chlorhexidine compound, such as chlorhexidine gluconate (CHG) compound, an octenidine compound, an iodine based compound, or other known compounds to increase the effectiveness of the anti microbial barrier formed by the dressing 10 when applied around the exit site 12 of the patient. The absorbent layer 42 may be impregnated with the compound to slowly release it over time, keeping the exit site 12 sterile.

The exemplary dressing body 24 may have a diameter of about 2.0 cm to about 2.5 cm. However, while the shape of dressing body 24 is shown as being generally circular, the dressing body 24 may be formed having any desired shape and size configuration. It should be noted that incorporating different shapes and sizes allows the dressing 10 to more effectively accommodate various parts of the body, for example. Therefore, the present invention contemplates different shapes of the dressing body 24 to cover exit sites at various parts of the body, such as arms, chest, stomach, back, etc. For example, the dressing body 24 may be substantially oval, triangular, square, or formed into a fanciful design such as a heart, for example.

As briefly described above, the dressing 10 includes the clasp 28 disposed in the opening 26 formed in the dressing body 24. The clasp 28 is configured to engage the percutaneous device 14, such as the catheter line 20, that extends through the opening 26 to hold the dressing 10 in place over the exit site 12. As shown in FIGS. 2A-3, the clasp 28 is generally U-shaped and includes a bend portion 48 from which a pair of generally parallel legs 50 extend. The bend portion 48 of the clasp 28 is configured to be disposed within the opening 26 in the dressing body 24 and the legs 50 are configured to extend along the radial edges 34 that form the slot 30. The pair of legs 50 of the clasp 28 may extend the entire length of the slot 30 between the opening 26 and the periphery 32 of the dressing body 24, as shown in FIG. 2. Terminal ends of the legs 50 may be rounded or curved to match a curvature of the periphery 32 of the dressing body 24. Alternatively, the legs 50 of the clasp 28 may extend partially along the length of the slot 30, as shown in FIG. 3, terminating partway along the length of the slot 30. Terminal ends of the legs 50 may be squared or rounded. The legs 50 are configured to maintain the slot 30 in an open position while also supporting the catheter line 20 as it turns into the opening 26 and enters the patient's body at the exit site 12. To that end, the legs 50 of the clasp 28 may terminate adjacent to the opening 26, within a region along the slot 30 that is 50% or less of the length of the slot 30 measured from the opening 26.

The clasp 28 may be formed of plastic or metal, such as a medical grade plastic or a medical grade metal, for example. For instance, the clasp 28 may be formed of plastic such as Polycarbonate (PC), Polyethylene (PE), Polypropylene (PP), Polyvinyl Chloride (PVC), Polytetrafluoroethylene (PTFE/Teflon), and Polyetheretherketone (PEEK). Alternatively, the clasp 28 may be formed of metals such as Stainless Steel (316L), Titanium, Cobalt-Chromium Alloys, Nitinol (Nickel-Titanium Alloy), Platinum, and Tantalum.

FIG. 4A is a schematic cross-section of the dressing 10 taken along line 4A-4A of FIG. 1, illustrating additional details of the clasp 28 and the engagement between the clasp 28 and the catheter line 20 of the percutaneous device 14. As shown, the clasp 28 includes a web member 52 that extends between a top flange 54 and an opposite bottom flange 56. The top flange 54, bottom flange 56, and the web member 52 define an external facing or peripheral channel 58 that extends around the entirety of the clasp 28. As shown in FIG. 4A, the dressing body 24 is configured to be received into the channel 58, thereby securing the clasp 28 to the dressing body 24. Specifically, the dressing body 24 is configured to be sandwiched between the top flange 54 and the bottom flange 56 of the clasp 28. That is, both the film layer 44 and the absorbent layer 42 are positioned between the top flange 54 and the bottom flange 56 of the clasp 28. In this configuration, the top flange 54 of the clasp 28 engages the top surface 38 of the dressing body 24, while the bottom flange 56 engages the bottom surface 40. The top and bottom flanges 54, 56 may be tapered to form a smooth transition between the dressing body 24 and the clasp 28, for example. The web member 52 of the clasp 28 is configured to engage the radial edges 34 of the dressing body 24 and the inner sidewall 36 of the dressing body 24. To that end, the fit between the channel 58 of the clasp 28 and the dressing body 24 may a frictional fit, ensuring that the clasp 28 is securely attached to the dressing body 24. As shown in FIG. 4A, the clasp 28, and in particular the bend portion 48 and the legs 50 of the clasp 28, is configured to engage the catheter line 20 to couple the dressing 10 to the percutaneous device 14.

The catheter line 20 is configured to be engaged by the bend potion 48 of the clasp 28 once the dressing 10 has been applied over the exit site 12 of the percutaneous device 14. To facilitate the engagement between the clasp 28 and the catheter line 20, the bend portion 48 of the clasp 28 may extend circumferentially for more than 180° about the opening 26, and may extend circumferentially for 270° or more about the opening 26 in the dressing body 24. In the exemplary embodiment shown, the bend portion 48 of the clasp 28 extends circumferentially for approximately 270° about the opening 26 in the dressing body 24. As a result, the bend portion 48 defines a pair of shoulders 60 where the bend potion 48 of the clasp 28 transitions to each leg 50, as shown in FIGS. 2-3.

The bend portion 48 of the clasp 28 is configured to extend approximately 270° about the catheter line 20, with the shoulders 60 being configured to prevent the catheter line 20 from inadvertently releasing or decoupling from the bend portion 48 of the clasp 28. In that regard, the fit between the bend portion 48 of the clasp 28 and the catheter line 20 may be a snap fit. As a result of the width of the slot 30 being less than the diameter of the catheter line 20, the width of the slot 30 and the legs 50 of the clasp 28 may spread apart to receive the catheter line 20 into the dressing 10 along the slot 30. Once the catheter line 20 is received into the bend portion 48 of the clasp 28, the legs 50 may be released to bias back to their normal positions, thereby capturing and retaining the catheter line 20 in the bend portion 48 of the clasp 28 and thus the opening 26. To that end, the bend portion 48 of the clasp 28 is configured to directly engage the catheter line 20. In particular, the bend portion 48 of the clasp 28 may apply a force normal to the catheter line 20 to grip the catheter line 20, thereby preventing the dressing 10 from twisting or moving along the catheter line 20.

A method of applying the dressing 10 over an exit site 12 of a percutaneous device 14 extending from the skin 16 of a patient will now be described. In that regard, the percutaneous device 14, and in particular the catheter line 20 of the percutaneous device 14 may be inserted into the body of the patient at the exit site 12. The dressing 10 may be removed from its packaging, for example, for installation to the percutaneous device 14 and over the exit site 12. The dressing 10, and in particular the dressing body 24 may be gripped with both hands adjacent to or along the legs 50 of the clasp 28 to slightly spread the legs 50 apart to receive or position the catheter line 20 into the slot 30 at the periphery 32 of the dressing 10. Next, advancing the catheter line 20 from the periphery 32 of the dressing 10 along the slot 30 in a direction toward to the opening 26 in the dressing 10. That is, moving or sliding the dressing 10 relative to the catheter line 20, while keeping the legs 50 of the clasp 28 slightly spread apart, to advance the catheter line 20 along the slot 30 and the legs 50 of the clasp 28 toward the bend portion 48 of the clasp 28 and the opening 26. Once the catheter line 20 is received into the bend portion 48 of the clasp 28, the legs 50 and dressing body 24 of the dressing 10 may be released to couple the dressing 10 to the catheter line 20 of the percutaneous device 14. Once released, the legs 50 of the clasp 28 are biased or spring back to their normal position, thereby capturing and retaining the catheter line 20 in the clasp 28 and thus the opening 26. At this point, the dressing 10 is considered coupled to the percutaneous device 14. The clasp 28 engages the catheter line 20, applying a slight gripping or clamping force to maintain a secure connection, ensuring the dressing 10 remains positioned over the exit site 12 and relative to the percutaneous device 14.

With reference to FIGS. 1 and 4A, the pair of legs 50 of the clasp 28 are configured to straddle the catheter line 20. As shown, the catheter line 20 is configured to extend over the dressing body 24 along the slot 30 to the opening 26 where the catheter line 20 turns in to the exit site 12. The catheter line 20 may be cradled by the legs 50 of the clasp 28, and in particular the top flange 54 of each leg 50 of the clasp 28, such that a portion of the catheter line 20 is recessed or received into the slot 30 to maintain a position of the catheter line 20 over the dressing 10. In particular, once the catheter line 20 is received into the bend portion 48 of the clasp 28, and the legs 50 and dressing body 24 of the dressing 10 are released to couple the dressing 10 to the catheter line 20 of the percutaneous device 14, the legs 50 of the clasp 28 may engage the portion of the catheter line 20 that extends along dressing 10 between the periphery 32 and the opening 26. This engagement between the legs 50 of the clasp 28 and the catheter line 20 further serves to prevent the movement of the dressing 10 relative to the catheter line 20, such as rotational movement.

The dressing 10 is configured to be installed such that the dressing body 24 is in contact with the skin 16 of the patient. Specifically, the bottom surface 40 defined by the absorbent layer 42 is configured to contact the patient's skin 16. The absorbent layer 42 may extend circumferentially about the exit site 12, as well as partially over the exit site 12. As described above, the dressing 10, and in particular the bottom surface 40 of the dressing 10 is non-adhesive and thus is not adhered to the skin 16 of the patient. Rather, the positioning of the dressing 10 on the patient's skin 16 and about the exit site 12 is maintained as a result of the engagement between the clasp 28 and the catheter line 20. While in the dressing 10 is installed, the absorbent layer 42 may slowly release antiseptic and/or antimicrobial compound onto the patient's skin 16 and the exit site 12 to keep the exit site 12 and skin 16 therearound sterile.

To remove the dressing 10 from the patient, the dressing body 24 may be gripped with both hands adjacent to or along the legs 50 of the clasp 28 to slightly spread the legs 50 apart enough to release the catheter line 20 from the bend portion 48 of the clasp 28. Once released, the dressing 10 may be slid or moved to advance the catheter line 20 along the slot 30 and the legs 50 of the clasp 28, away from the bend portion 48 of the clasp 28 and the opening 26 in the dressing body 24. By continuing to pull the dressing 10 away from the catheter line 20, the catheter line 20 will eventually exit the slot 30 at the periphery 32 of the dressing 10. The dressing 10 may then be appropriately discarded.

Turning now with reference to FIG. 4B, where like reference numerals represent like features compared to the embodiment of the dressing 10 described above with respect to FIGS. 1-4A, the dressing 10 is shown in accordance with another embodiment of the present invention. As shown, the absorbent layer 42 of the dressing body 24 is received into the peripheral channel 58 of the clasp 28 to secure the clasp 28 to the dressing body 24. In this configuration, the top flange 54 of the clasp 28 engages the top surface 46 of the absorbent layer 42 while the bottom flange 56 engages the bottom surface 40 of the dressing 10, being the bottom surface of the absorbent layer 42. The film layer 44 is disposed on the top surface 38 of the absorbent layer 42 and over the top flange 54 of the clasp 28. The film layer 44 may extend over the top flange 54 of the clasp 28 and terminate along the top surface 38 of the dressing body 24 at the opening 26 and along the slot 30. As shown, this results in a generally smooth and uninterrupted top surface 38 of the dressing body 24.

Referring now to FIG. 4C, where like reference numerals represent like features compared to the embodiments of the dressing 10 described above with respect to FIGS. 1-4B, the dressing 10 is shown in accordance with another embodiment of the present invention. The embodiment shown in FIG. 4C is similar in may respects to the embodiment shown in FIG. 4B, except that rather than terminating along the top surface 38 of the dressing body 24 at the opening 26 and along the slot 30, the film layer 44 extends into the opening 26 and the slot 30. That is, the film layer 44 may extend over the top flange 54 and over the web member 52, terminating generally at the bottom surface 40 of the dressing body 24. As a result, the film layer 44 is positioned between the clasp 28 and the catheter line 20 such that the film layer 44 contacts the catheter line 20 when the dressing 10 is installed to the percutaneous device 14. To that end, the film layer 44 may provide the clasp 28 with additional grip, thereby improving the attachment of the dressing 10 to the catheter line 20.

Referring now to FIG. 5A, where like reference numerals represent like features compared to the embodiments of the dressing 10 described above with respect to FIGS. 1-4C, the dressing 10 is shown in accordance with another embodiment of the present invention. As shown, the absorbent layer 42 of the dressing body 24 is received into the peripheral channel 58 of the clasp 28 to secure the clasp 28 to the dressing body 24. In this configuration, the absorbent layer 42 includes a male projection or tongue 62 that is configured to be received into the peripheral channel 58 of the clasp 28. The tongue 62 may be a continuous projection that extends along the radial edges 34 and the inner sidewall 36 of the dressing body 24. As shown, the tongue 62 is sandwiched between the top flange 54 and the bottom flange 56 of the clasp 28. The top flange 54 of the clasp 28 extends over the tongue 62 so as to be generally flush with the top surface 46 of the absorbent layer 42. Similarly, the bottom flange 56 of the clasp 28 extends of the tongue 62 so as to be generally flush with the bottom surface 40 of the dressing body 24. The film layer 44 extends over the top flange 54 of the clasp 28 and terminates at the opening 26 and along the slot 30. As shown, this results in a generally smooth and uninterrupted top surface 38 and bottom surface 40 of the dressing body 24. In this embodiment, the film layer 44 and the clasp 28 may engage the catheter line 20 when the dressing 10 is installed to the percutaneous device 14.

Referring now to FIG. 5B, where like reference numerals represent like features compared to the embodiments of the dressing 10 described above with respect to FIGS. 1-5A, the dressing 10 is shown in accordance with another embodiment of the present invention. The embodiment shown in FIG. 5B is similar in may respects to the embodiment shown in FIG. 5A, except that rather than terminating at the opening 26 and along the slot 30, the film layer 44 extends into the opening 26 and the slot 30. That is, the film layer 44 may extend over the top flange 54 and over the web member 52, terminating generally at the bottom surface 40 of the dressing 10. As a result, the film layer 44 is configured to be positioned between the clasp 28 and the catheter line 20 such that the film layer 44 contacts the catheter line 20 when the dressing 10 is installed to the percutaneous device 14. To that end, the film layer 44 may provide the clasp 28 with additional grip, thereby improving the attachment of the dressing 10 to the catheter line 20.

While the present invention has been illustrated by the description of various embodiments thereof, and while the embodiments have been described in considerable detail, it is not intended to restrict or in any way limit the scope of the appended claims to such detail. Thus, the various features discussed herein may be used alone or in any combination. Additional advantages and modifications will readily appear to those skilled in the art. The invention in its broader aspects is therefore not limited to the specific details and illustrative examples shown and described. Accordingly, departures may be made from such details without departing from the scope of the general inventive concept.

What is claimed is:

1. A dressing for use at an exit site of a percutaneous device that extends from skin of a patient, the dressing comprising:
a dressing body including a periphery, a top surface, an opposite bottom surface that is configured to contact the skin of the patient, an opening configured to receive a portion of the percutaneous device through the dressing body, and a slot that extends from the opening to the periphery of the dressing body, the dressing body including an absorbent layer impregnated with an antiseptic compound; and
a clasp disposed in the opening, the clasp being configured to engage the portion of the percutaneous device that extends through the opening to hold the dressing in place over the exit site.

2. The dressing of claim 1, wherein the bottom surface of the dressing body is non-adhesive.

3. The dressing of claim 1, wherein the clasp extends about an inner wall of the dressing body defined by the opening.

4. The dressing of claim 1, wherein the clasp is configured to directly contact the portion of the percutaneous device that extends through the opening.

5. The dressing of claim 4, wherein the clasp is configured to apply a force so as to grip the portion of the percutaneous device that extends through the opening.

6. The dressing of claim 3, wherein the clasp includes a pair of legs, with each leg being configured to extend partially along the slot in the dressing body.

7. The dressing of claim 6, wherein the pair of legs are spaced apart from each other to define an open space therebetween.

8. The dressing of claim 7, wherein the space between the pair of legs is constant along a length of the slot.

9. The dressing of claim 6, wherein each leg includes a web member that extends between a top flange and a bottom flange, each web member being configured to engage a radial edge that defines the slot in the dressing body.

10. The dressing of claim 7, wherein the percutaneous device includes a catheter line, and the pair of legs are configured to straddle the catheter line.

11. The dressing of claim 1, wherein the dressing body further comprises a film layer disposed on top of the absorbent layer, the film layer forming the top surface of the dressing body.

12. The dressing of claim 11, wherein the film layer extends into the opening such that the film layer is configured to be arranged between the clasp and the portion of the percutaneous device that extends through the opening.

13. The dressing of claim 1, wherein the clasp is coupled to the dressing body such that the top surface and the bottom surface of the dressing body are generally planar.

14. The dressing of claim 13, wherein the clasp is coupled to the absorbent layer.

15. The dressing of claim 1, wherein the clasp includes a web member that extends between a top flange and a bottom flange to form a channel, the dressing body being received within the channel to couple the clasp to the dressing body.

16. The dressing of claim 15, wherein the top flange of the clasp is configured to engage the top surface of the dressing body and the bottom flange is configured to engage the bottom surface of the dressing body.

17. The dressing of claim 1, wherein the antiseptic compound includes Chlorhexidine.

18. The dressing of claim 1, wherein a thickness measured between the top surface and the bottom surface of the dressing body is within a range of between about 0.1 cm to about 0.3 cm.

19. A method of applying a dressing about an exit site of a percutaneous device that extends from skin of a patient, the method comprising:
providing the dressing, comprising:
a dressing body including a periphery, a top surface, an opposite bottom surface that is configured to contact the skin of the patient, an opening configured to receive a portion of the percutaneous device through the dressing body, and a slot that extends from the opening to the periphery of the dressing body, the dressing body including an absorbent layer impregnated with an antiseptic compound; and
a clasp disposed in the opening;
positioning the percutaneous device in the slot;
moving the dressing relative to the percutaneous device to receive the percutaneous device into the slot in a direction toward the opening in the dressing;
receiving the percutaneous device within the clasp;
positioning the bottom surface of the dressing body into engagement with the skin of the patient surrounding the exit site; and
engaging the percutaneous device with the clasp to hold the dressing in place over the exit site.

20. The method of claim 19, wherein the clasp includes a pair of legs, and the slot in the dressing body is defined by a pair of radial edges, each leg being configured to extend partially along one radial edge of the dressing body, the method further comprising:
engaging the percutaneous device with the pair of legs to hold the dressing in place over the exit site.
